(19) United States
(12) Patent Application Publication
(10) Pub. No.: US 2024/0138429 A1
(43) Pub. Date: May 2, 2024

(54) Process for adjusting the characteristics of a starter culture

(71) Applicant: DIL Deutsches Institut fiir Lebensmitteltechnik e.V., Quakenbrück (DE)

(72) Inventors: Christian Hertel, Quakenbrück (DE); Panagiotis Chanos, Quakenbrück (DE); Corinna Stühmeier-Niehe, Quakenbrück (DE)

(21) Appl. No.: 18/494,913

(22) Filed: Oct. 26, 2023

(30) Foreign Application Priority Data
Oct. 27, 2022 (DE) 102022211432.9

(51) Int. Cl. A23C 9/123; A23C 19/068; C12N 1/20

(52) U.S. Cl. CPC A23C 9/1238; A23C 9/1234; A23C 19/0684; C12N 1/20; A23V 2400/137; A23V 2400/249

(57) ABSTRACT

Process for the production of starter cultures for the production of dairy products having predetermined properties, wherein the starter cultures are treated with pulsed electric fields (PEF) of rectangular monopolar high voltage pulses of a field strength of 0.2 to 1 kV/cm with a pulse duration of 5 to 8 μs at a frequency of 1 to 21 Hz for 20 to 80 pulses, for each parameter respectively ±15% or ±10% or ±5%.

The present application claims priority of the German patent application 10 2022 211 432.9, filed on 27 Oct. 2022.

The present invention relates to a method for adjusting the properties of starter cultures in the production of dairy products by fermentation of milk. Starter cultures comprising or consisting of lactic acid bacteria are adjusted by the process with respect to the properties they have on the product properties of dairy products in the production thereof, in particular on the firmness and/or syneresis, for example, of sour milk, yogurt and kefir products, e.g., yogurt, kefir, and cheese, e.g., fresh cheese, pasta filata, mozzarella. Accordingly, the invention relates to a process for producing starter cultures for the production of dairy products, and to a method for producing dairy products with the starter cultures produced or adjusted by the method, and to the use of the starter cultures produced by the method for producing dairy products by fermentation.

For the purposes of the invention, starter cultures may include, in particular, Streptococcus, e.g. Streptococcus thermophilus, Lactobacillus, e.g. Lactobacillus delbruekii ssp. bulgaricus, Lactobacillus delbruekii ssp. lactis, Lactobacillus helveticus, Lactobacillus acidophilus, Lactobacillus casei, Lactobacillus kefir, Lactobacillus parakefir, Lactococcus, e.g. Lactococcus lactis ssp. lactis, Lactococcus lactis ssp. lactis biovar. diacetylactis, Lactococcus lactis ssp. cremoris, Leuconostoc, e.g. Leuconostoc mesenteroides ssp. cremoris, Bifidobacterium, e.g., Bifidobacterium lactis, and mixtures of at least two of these, preferably a mixture of Lactobacillus delbruckii ssp. bulgaricus and Streptococcus thermophilus. The process has the advantage that the starter cultures can be adjusted to control, in particular to enhance or attenuate, predetermined product properties of the dairy products in the fermentation of the production of dairy products.

Chanos et al, European Food Research and Technology (2020), 246: 621-630 describes the treatment of the lactic acid bacteria Streptococcus thermophilus and Lactobacillus delbrüekii ssp. bulgaricus with pulsed electric fields (PEF) selected from a combination of an electric field strength of 1 or 3.67 kV/cm, a frequency of 0.5 or 4 Hz, and a pulse number of 5 or 50, and exhibiting an exponential voltage waveform due to the ELCRACK HPV system used (Elea Vertriebs- und Vermarktungsgesellschaft mbH, Quakenbrück, Germany). The optimal treatment was PEF at 4 Hz, 1 kV/cm for 50 pulses and resulted in a shortening of the lag time in yogurt fermentation of 12 min. For certain PEF parameters, a 50% smaller size of the inoculum of the starter culture could be used and an acceleration of acidification was observed.

The patent DE 10 2012 201 822 B4 for the production of starter cultures with lactic acid bacteria describes their treatment with pulsed electric fields of a field strength of 7.5 to 10 kV/cm in order to achieve a stronger acidification during the ripening of raw sausage.

The invention has the object of providing an alternative process for treating starter cultures, in particular for adjusting their fermentation properties for dairy products, and of specifying a process for the production of dairy products in which the product properties are predetermined by adjusting the starter cultures. Preferably, the starter cultures should result in a milk product with low syneresis and high firmness.

The invention archives the object by the features of the claims and in particular provides a process for the production of starter cultures which are adjusted for the production of dairy products with predetermined properties in that the starter cultures are treated by pulsed electric fields (PEF), consisting of rectangular monopolar high voltage pulses of a field strength of 0.2 to 1 kV/cm with a pulse duration of 5 to 8 μs at a frequency of 1 to 21 Hz, for 20 to 80 pulses, for each parameter respectively ±15% or ±10% or ±5%, e.g. a field strength of 0.6 kV/cm with a pulse duration of 7 μs at a frequency of 11 Hz for 50 pulses, for each parameter respectively ±25% or ±20% or ±15% or ±10%. During treatment with PEF, the starter cultures may be suspended in aqueous medium, e.g., peptone water, milk, water, or isotonic aqueous solution. Optionally, the starter cultures are frozen after treatment with PEF in the same medium or without changing the medium, preferably immediately after treatment with PEF, and in particular without adding an antifreeze.

The predetermined properties of the dairy products produced with the starter cultures are selected from syneresis, which is preferably lower than that produced with a starter culture of the same bacteria not treated by PEF, firmness, which is higher or lower than that produced with a starter culture of the same bacteria not treated by PEF, and/or taste. Preferably, the dairy product is yogurt or cheese, in particular mozzarella. Accordingly, the invention relates to a method for producing starter cultures for use in the production of dairy products, as well as to a method for producing dairy products, wherein the dairy products preferably have a higher strength and/or a lower syneresis, e.g. compared to dairy products produced under the same conditions, but with the same bacteria as starter cultures, optionally frozen, but not treated with PEF. In this case, the dairy product is preferably yogurt or cheese, in particular mozzarella. In the embodiment where the dairy product is mozzarella, the dairy product may optionally have a lower firmness and/or a less cheesy and/or less sour taste.

It has been shown that by treating starter cultures by means of these pulsed electric fields (PEF treatment) during the fermentation of milk, yogurt is produced that has higher strength and/or lower syneresis compared to a starter culture of the same bacteria but without this PEF treatment. The lag phase during acidification and the achieved acidification were not or only slightly affected by the PEF treatment and corresponded to the lag phase and achieved acidification obtained during fermentation with not the same bacteria without prior PEF treatment.

For the production of yogurt for PEF treatment of the starter culture, pulsed electric fields of 0.2 kV/cm, 20 pulses at 21 Hz, 8 μs pulse duration (PEF4), or 0.2 kV/cm, 80 pulses at 1 Hz, 5 μs pulse duration (PEF5), or 0.2 kV/cm for PEF treatment of the starter culture, 80 pulses at 1 Hz, pulse duration 8 μs (PEF6), or 0.2 kV/cm, 20 pulses at 1 Hz, 8 μs pulse duration (PEF2), or 1 kV/cm, 80 pulses at 21 Hz, 8 μs pulse duration (PEF16), in each case consisting of rectangular, monopolar high-voltage pulses are preferred. In milk fermentation for the production of yogurt, starter cultures produced with pulsed electric fields PEF5 give a shorter lag phase, with PEF6 yogurt with a lower syneresis, with PEF4 by higher proteolytic activity yogurt with increased content of free amino acids, with PEF2 yogurt with greater firmness. These properties of the starter cultures were retained even after freezing in liquid nitrogen and subsequent storage at −20° C. for 2 weeks and produced comparable or preferably the same product properties in the yogurt.

For the production of cheese, e.g. mozzarella, PEF treatment of the starter culture with pulsed electric fields of 0.2 kV/cm, 20 pulses at 21 Hz, 8 μs pulse duration (PEF4), or 0.2 kV/cm, 80 pulses at 1 Hz, 5 μs pulse duration (PEF5), or 0.2 kV/cm, 80 pulses at 1 Hz, 8 μs pulse duration (PEF6), or 0.2 kV/cm, 20 pulses at 1 Hz, 8 μs pulse duration (PEF2) is preferred. Compared to a starter culture not treated with PEF, a starter culture treated with PEF2 yields a firmer mozzarella with a milder, less acidic and less cheesy taste, a starter culture treated with PEF4 yields a more acidic taste, and a starter culture treated with PEF5 yields a firmer mozzarella with substantially unchanged flavor, a starter culture treated with PEF6 yields a softer mozzarella with substantially unchanged taste.

The starter cultures treated with the process have the advantage of producing higher firmness in the yogurt during the fermentation of milk for the production of yogurt, preferably additionally lower syneresis.

Furthermore, the starter cultures treated with the process have the advantage that the fermentation of yogurt or cheese, e.g. mozzarella, carried out with them have a shorter lag phase compared to untreated starter cultures, e.g. shorter by at least 20 min, preferably by at least 25 min.

In general, it has shown that the starter cultures produced with the PEF treatment retain substantially the ability to produce the product characteristics of dairy products produced therewith for predetermined firmness and/or lower syneresis even after freezing and/or freeze-drying. Therefore, after PEF treatment, the starter cultures may be frozen or freeze-dried, stored, preferably at −20° C. or below, e.g., in liquid nitrogen, and subsequently used to produce dairy products. During freezing and storage, an antifreeze agent can be added to the starter culture before or after PEF treatment, e.g. glycerol.

The invention is now described by means of examples with reference to the figures which show in

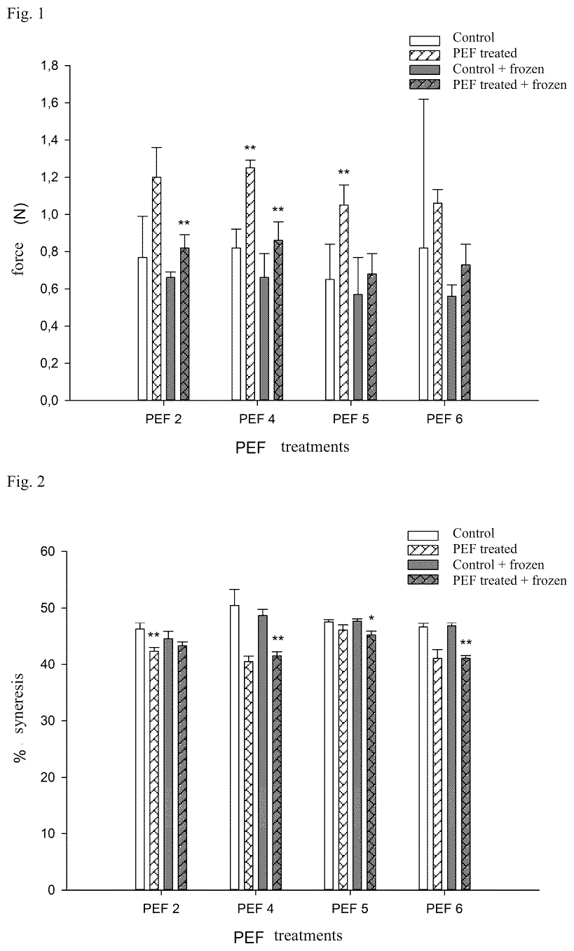

FIG. 1 strength values for yogurt,

FIG. 2 values for the syneresis of yogurt,

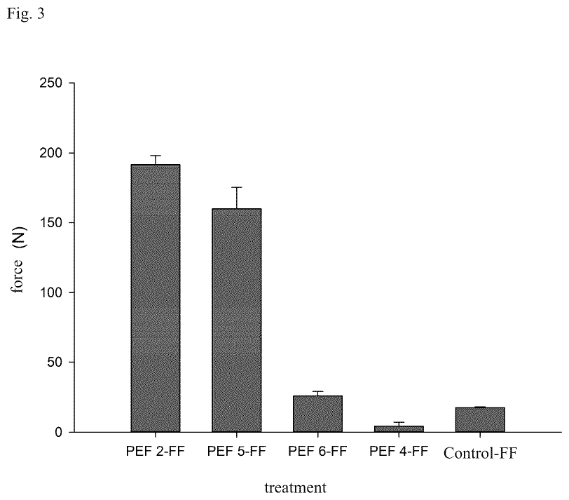

FIG. 3 strength values for mozzarella

### EXAMPLE 1: PROCESS FOR THE PRODUCTION OF STARTER CULTURES FOR YOGURT

As starter cultures for yogurt, Lactobacillus delbrueckii subsp. bulgaricus, strain LB186 were incubated separately at 37° C. in mMRS broth, and Streptococcus thermophilus, strain ST504, were incubated separately at 42° C. in M17 broth with agitation (50 rpm) until an OD₆₀₀ of 0.4 was reached, with these inoculating separate media in each case and incubating for approx. 8 h incubated until final concentrations of approximately 3×10⁸ CFU/ml were reached. These cultures were centrifuged separately at 10000×g, washed twice in sterile peptone water, and then suspended in peptone water and mixed in equal parts to give a cell count of 2.5×10⁸ CFU/ml. This suspension was exposed to pulsed electric fields consisting of monopolar rectangular high-voltage pulses, which were varied in the following PEF parameters, between parallel electrodes (PEF-Pilot Dual system, Elea Vertriebs- und Vermarktungs-GmbH, Quakenbrück, Germany) spaced 20 cm apart without agitation of the suspension:
Voltage: 4 kV (0.2 kV/cm), 20 kV (1 kV/cm) or 12 kV (0.6 kV/cm),
Pulse count: 20 or 80 or 50,
Frequency: 1 Hz, 21 Hz, or 11 Hz,
Pulse duration: 5 μs, 8 μs, or 7 μs.

Starter cultures prepared in this way were either mixed directly into milk or frozen in liquid nitrogen without additive, optionally with addition of an antifreeze, stored at −20° C. and thawed (“frozen”) and subsequently mixed into milk. Starter cultures that were not treated with PEF and optionally frozen were included as control samples in parallel experiments.

Milk was 9 wt % skim milk powder (SMP SH Basic Skim Milk Powder, DMK GmbH, Zeven) dissolved in distilled water, heated to 90° C. and stored at 4° C. for 1 d before addition of the starter culture.

Individual beakers were filled with 147 ml of the milk, which was preheated to 42° C., and a starter culture was added to each to a final concentration of 5×10⁶ CFU/ml. The beakers were sealed with an airtight lid through which a pH probe protruded into the milk. The beakers, parallel sets for each analysis, were incubated statically at 42° C., avoiding temperature differences between individual beakers.

The course of acidification, measured as change in pH, showed no significant dependence on any of the PEF parameters.

It was found that PEF treatment significantly affected the firmness of yogurt.

| | | |
|---|---|---|
| PEF parameters | Syneresis | Strength |

0.2 kV/cm, 80 pulses at 1 Hz, pulse duration 5 μs (PEF5)

| | | |
|---|---|---|
| Control | 47.57 ± 0.40 | 0.65 ± 0.19 |
| PEF treated | 46.05 ± 0.91 | 1.05 ± 0.11 |
| Control + frozen | 47.70 ± 0.41 | 0.57 ± 0.20 |
| PEF treated + frozen | 45.15 ± 0.66 | 0.68 ± 0.11 |

0.2 kV/cm, 20 pulses at 1 Hz, 8 μs pulse duration (PEF2)

| | | |
|---|---|---|
| Control | 46.21 ± 1.13 | 0.77 ± 0.22 |
| PEF treated | 42.35 ± 0.68 | 1.20 ± 0.16 |
| Control + frozen | 44.52 ± 1.31 | 0.66 ± 0.03 |
| PEF treated + frozen | 43.29 ± 0.71 | 0.82 ± 0.07 |

0.2 kV/cm, 20 pulses at 21 Hz, 8 μs pulse duration (PEF4)

| | | |
|---|---|---|
| Control | 50.48 ± 2.77 | 0.82 ± 0.10 |
| PEF treated | 40.54 ± 0.95 | 1.25 ± 0.04 |
| Control + frozen | 48.69 ± 1.09 | 0.66 ± 0.13 |
| PEF treated + frozen | 41.55 ± 0.70 | 0.86 ± 0.10 |

0.2 kV/cm, 80 pulses at 1 Hz, pulse duration 8 μs (PEF6)

| | | |
|---|---|---|
| Control | 46.58 ± 0.70 | 0.82 ± 0.8 |
| PEF treated | 41.10 ± 1.54 | 1.06 ± 0.073 |
| Control + frozen | 46.79 ± 0.57 | 0.56 ± 0.06 |
| PEF treated + frozen | 41.11 ± 0.47 | 0.73 ± 0.11 |

1 kV/cm, 80 pulses at 21 Hz, 8 μs pulse duration (PEF16)

| | | |
|---|---|---|
| PEF treated | 36.78 ± 9.01 | 1.42 ± 0.47 |

The data are mean values of syneresis and maximum strength values. Strength is generally given in N, measured as maximum force (N) for penetration of a cylindrical probe, diameter 36 mm, penetration depth 10 mm, feed rate 0.5 mm/s, load 1 N (Ta.XT plus Texture Analyzer, Stable Microsystems, Goldaming, GB).

Syneresis is generally expressed as % liquid released, measured by storing the sample at −20° C. for a maximum of 7 days, thawing at room temperature for 3 h, centrifuging at 500×g for 10 min at 4° C., and weighing the liquid released. The syneresis indicates the proportional weight of released liquid to the remaining yogurt.

FIG. 1 shows strength values of yogurt for different PEF treatments used in the production of the starter cultures. Only the values shown in each group can be compared with each other.

The values show that treating the starter cultures with PEF2, PEF4, PEF5 and PEF6 and then mixing them directly into milk (PEF treated) resulted in the production of yogurt of higher strength than with untreated starter culture (Control), and yogurt of higher strength was also produced when frozen after PEF treatment (PEF treated+frozen) and subsequently mixed into milk than with starter cultures without PEF treatment that were frozen in the same way (Control+frozen).

FIG. 2 shows the influence of PEF treatment on syneresis, determined as the release of liquid from yogurt. The values show that the treatment of starter cultures with PEF2, PEF4, PEF5 and PEF6 after direct mixing into milk (PEF treated) resulted in the production of yogurt of lower syneresis than with untreated starter culture (Control), and also when frozen after PEF treatment (PEF treated+frozen) yogurt of lower syneresis was produced than with starter cultures without PEF treatment, which were equally frozen (Control+frozen).

The results show that PEF treatment produces starter cultures that favorably affect firmness (higher) and syneresis (lower) in the production of dairy products using yogurt as an example. These properties of the starter cultures produced by PEF treatment were retained even after freezing and storage at −20° C. for 14 d.

Furthermore, it was found that treatment with PEF5 produces starter cultures that have a shorter lag phase during fermentation compared to untreated starter cultures by at least 20 min, preferably by 21 min.

### Example 2: Process for the Production of Starter Cultures for Mozzarella Cheese

The starter cultures were prepared according to Example 1.

Cylindrical mozzarella samples were stored at room temperature for at least 30 min before starting the measurement. The cylindrical probe (36 mm diameter) of the Ta.XT plus Texture Analyzer moved at a speed of 1 mm/s over a distance of 8 mm.

The results presented in FIG. 3 showed that the firmness of mozzarella cheese prepared with PEF-treated cultures was significantly different from the firmness of mozzarella cheese prepared with the non-treated starter culture (Control). While mozzarellas prepared with PEF 2-FF, PEF 5-FF and PEF 6-FF cultures had higher firmness than those prepared with the Control culture, the starter culture treated with PEF4 produced softer mozzarella than the Control culture. These results are consistent with the sensory evaluation of firmness, although mozzarella prepared with PEF6-treated starter culture when measured was also significantly firmer than that prepared with Control culture.

Mozzarella produced with a starter culture treated with PEF2 was evaluated as milder (less acidic/cheesy) compared to the control. In contrast, mozzarella produced with a starter culture treated with PEF4 was evaluated as more acidic than the control. No significant differences in taste were found for mozzarellas produced with starter cultures treated by PEF 5 or PEF 6 compared to the control FF. Regarding perceived firmness/chewiness, starter cultures treated with PEF 2-FF and PEF 5-FF yielded significantly firmer/chewier mozzarellas than the control. In contrast, a starter culture treated with PEF 4 yielded softer/less chewy mozzarella than the control. Starter culture treated with PEF 6 produced no significant differences in firmness and chewiness from the control.

This result shows that the production of starter cultures with treatment by pulsed electric fields produces properties of the starter cultures that specifically lead to other firmnesses and optionally less cheesy taste in the production of cheese, here represented by mozzarella.

1. Process for the production of starter cultures for use in the manufacture of dairy products by treating starter cultures with pulsed electric fields (PEF) consisting of rectangular monopolar high voltage pulses of a field strength of 0.2 to 1 kV/cm with a pulse duration of 5 to 8 μs at a frequency of 1 to 21 Hz, ±15% for each parameter, for 20 to 80 pulses.

2. Process according to claim 1, characterized in that after treatment with pulsed electric fields the starter cultures are frozen or freeze-dried.

3. Process according to claim 1, characterized in that the starter culture is selected from Streptococcus thermophilus, Lactobacillus, e.g. Lactobacillus delbruckii ssp. bulgaricus. Lactococcus, e.g. Lactococcus lactis ssp. lactis, Leuconostoc, e.g. Leuconostoc cremoris, Bifidobacterium, e.g. Bifidobacterium lactis, and mixtures of at least two of these.

4. Process according to claim 1, characterized in that the treatment with PEF occurs at 0.2 kV/cm, 20 pulses at 21 Hz, 8 μs pulse duration (PEF4), or 0.2 kV/cm, 80 pulses at 1 Hz, pulse duration 5 μs (PEF5), or 0.2 kV/cm, 80 pulses at 1 Hz, pulse duration 8 μs (PEF6), or 0.2 kV/cm, 20 pulses at 1 Hz, 8 μs pulse duration (PEF2), or 1 kV/cm, 80 pulses at 21 Hz, 8 μs pulse duration (PEF16), for each parameter ±15% in each case.

5. Process for preparing a dairy product by fermentation of milk with a starter culture, characterized in that the starter culture is produced by a process according to claim 1.

6. Process according to claim 5, characterized in that the dairy product is yogurt, wherein yogurt has a higher strength and a lower syneresis due to the treatment of the starter cultures with PEF than yogurt produced under otherwise identical conditions with non-PEF-treated starter cultures.

7. Process according to claim 5, characterized in that the dairy product is mozzarella, wherein mozzarella has a higher firmness due to the treatment of the starter cultures with PEF than mozzarella produced under otherwise identical conditions with non-PEF-treated starter cultures.

8. Process according to claim 5, characterized in that the milk is produced by dissolving milk powder in water.

9. Process according of claim 5, characterized in that the treatment is performed with PEF at 0.2 kV/cm, 20 pulses at 21 Hz, 8 μs pulse duration (PEF4), or 0.2 kV/cm, 80 pulses at 1 Hz, 5 μs pulse duration (PEF5), or 0.2 kV/cm, 80 pulses at 1 Hz, pulse duration 8 μs (PEF6), or 0.2 kV/cm, 20 pulses at 1 Hz, 8 μs pulse duration (PEF2), or 1 kV/cm, 80 pulses at 21 Hz, 8 μs pulse duration (PEF16), for each parameter 15% in each case, and that the milk product is yogurt.

10. Process according to claim 5, characterized in that the treatment is with PEF at 0.2 kV/cm, 20 pulses at 21 Hz, 8 μs pulse duration (PEF4), or 0.2 kV/cm, 80 pulses at 1 Hz, 5 μs pulse duration (PEF5), or 0.2 kV/cm, 80 pulses at 1 Hz, pulse duration 8 μs (PEF6), or 0.2 kV/cm, 20 pulses at 1 Hz, 8 μs pulse duration (PEF2), or 1 kV/cm, 80 pulses at 21 Hz, 8 μs pulse duration (PEF16), for each parameter 15% in each case, and that the dairy product is mozzarella.

11. Process according to claim 5, characterized in that the treatment with PEF occurs at 0.2 kV/cm, 20 pulses at 21 Hz, 8 μs pulse duration (PEF4), or 0.2 kV/cm, 80 pulses at 1 Hz, 5 μs pulse duration (PEF5), or 0.2 kV/cm, 80 pulses at 1 Hz, pulse duration 8 μs (PEF6), or 0.2 kV/cm, 20 pulses at 1 Hz, 8 μs pulse duration (PEF2), or 1 kV/cm, 80 pulses at 21 Hz, 8 μs pulse duration (PEF16), for each parameter ±15% in each case.
